(19) United States
(12) Patent Application Publication
(10) Pub. No.: US 2008/0319246 A1
(43) Pub. Date: Dec. 25, 2008

(54) Process for the Degradation and/or Detoxification of Chemical and Biological Pollutants

(76) Inventors: Giancarlo Cravotto, Collegno (IT); Wander Tumiatti, Collegno (IT); Carlo Maria Roggero, Collegno (IT)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/665,168

(22) PCT Filed: Oct. 12, 2005
(86) PCT No.: PCT/IB2005/003028

§ 371 Date: Jul. 25, 2008

(30) Foreign Application Priority Data
Oct. 15, 2004 (IT) MI2004A001966

(51) Int. Cl. A62D 3/13; A62D 3/176; A62D 3/178

(52) U.S. Cl. 588/304; 588/309; 588/310

(57) ABSTRACT

A process is described for the degradation, detoxification and decontamination treatment of contaminated solid or liquid matrices and/or relative functional revalorisation and for their sterilisation, which comprises placing said matrix in contact with at least one saline catalyst and at least one oxidising agent chosen from among percarbonates, perborates or metal peroxides in the presence of a last one energy source chosen from among microwaves (MW), ultrasounds (US) and ultraviolet rays (UV).

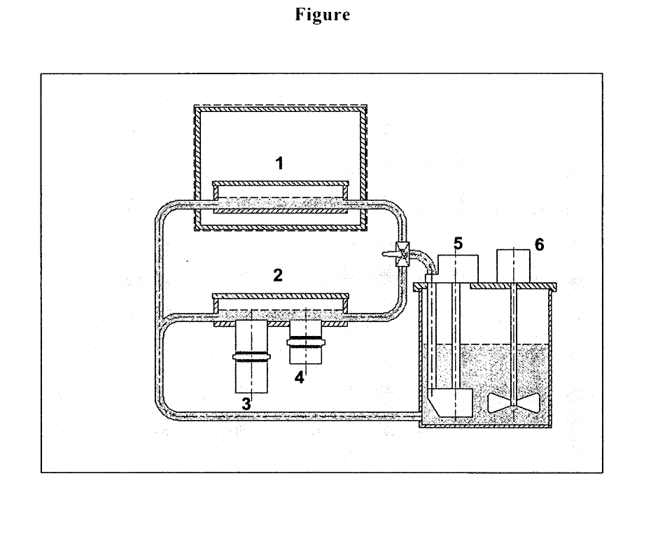

### FIELD OF THE INVENTION

The present invention relates to a chemical-physical process for the degradation, detoxification and decontamination treatment of solid or liquid matrices contaminated, in particular, by persistent organic pollutants (POPs), and/or for their related functional revalorisation and sterilisation.

The process of the invention is based on the synergy among oxidizing agents, saline catalysts and microwaves (MW) and/or high intensity ultrasounds (US) and/or ultraviolet rays (UV).

The process of the invention can also be used for the extraction of organometallic compounds (and possibly metals after the addition of chelants, acids and/or coadjuvants) from the same matrices.

This process allows a surprising reduction in treatment times, improving both the efficiency of oxidative degradation and the area of application, since it can be adapted to various substrates such as solid, liquid or gaseous matrices of a technical and environmental type.

### STATE OF THE ART

U.S. Pat. No. 6,663,781, U.S. Pat. No. 6,627,428, U.S. Pat. No. 36,082, DE10009894, U.S. Pat. No. 6,143,182, WO9921801, WO9521794 describe processes of oxidative degradation of chemical or biological pollutants, normally present in water or in soil, by means of Fenton's reagent (H₂O₂/Fe²⁺—hydrogen peroxide and ferrous sulphate). The use of 30% hydrogen peroxide as oxidant in the presence of iron salts (II) for the dehalogenation of haloarenes, chlorobenzoic acid and chlorophenols (preferably with acid pH) has also been widely described in literature. In particular a description was given of the efficacy of the treatment with Fenton's reagent in aqueous matrices or contaminated soils and simultaneous radiation with ultraviolet rays alone (Kusvuran E. et al., J. of Hazardous Materials 106 (2-3): 115-125, 2004; Pignatello Jj et al. Environmental Toxicology and Chemistry 13 (3): 423-427, 1994), with UV and ultrasounds (Gogate P R et al. Advances in Environmental Research 8 (3-4): 553-597, 2004; Wu C D et al., Water Research 35 (16): 3927-3933, 2001) with microwaves (Abramovitch R A et al., Chemosphere 50 (7): 955-957, 2003; Abramovitch R A et al., Chemosphere 38 (10): 2227-2236, 1999) or with ultrasounds (Neppolian B et al., Ultrasonics Sonochemistry 11 (5): 273-279, 2004; Shen Z Z et al., J. of Environmental Sciences-China, 16 (3): 431-435 2004; Emery R J et al., Environmental Technology 24 (12): 1491-1500, 2003; Jiang Y et al., Water Science and Technology 47 (10): 85-92, 2003; Nam S N et al., Ultrasonics Sonochemistry 10 (3): 139-147, 2003).

The use of percarbonates has been proposed above all for the degradation of industrial waste, in particular in the sectors of pigments and dyes (Ohura, R. et al., Textile Research Journal (1992), 62(9), 552-6; Miyamoto, T. Shizuoka-ken Hamamatsu Sen'i Kogyo Shikenjo Hokoku (1989), (28), 11-14. DE19644714 also describes the treatment of effluent resulting from the processing of metals with percarbonates, hydrogen peroxide, soda and surfactants in order to remove bactericides, biocides and other preservatives such as formaldehyde, phenols and their derivatives.

Heitkamp, M., Bioremediation J. (1997), 1(2), 105-114 describes a study of the microbial degradation of p-nitrophenol and phenol in the presence of agents releasing oxygen as percarbonates.

### DESCRIPTION OF THE INVENTION

The present invention relates to a chemical and physical process suitable for the functional, environmental and/or energy valorisation of solid and liquid matrices both from the biological standpoint, with the oxidative degradation of organic material and a broad spectrum germicidal action, and chemical one, with the degradation, detoxification and decontamination of organic and/or persistent compounds (POPs—Persistent Organic Pollutants), through methods involving synergy among oxidising agents, metal catalysts and microwaves (MW), high-intensity ultrasounds (US) and ultraviolet rays (UV) as sources of energy.

The process of the invention comprises the placing of said matrices in contact with at least one saline catalyst and at least one oxidising agent chosen from among percarbonates, perborates and metal peroxides, in the presence of at least one source of energy chosen from among microwaves (MW), ultrasounds (US) and ultraviolet rays (UV).

Said process allows in particular the degradation and/or detoxification of halogenated aromatic organic compounds (bromo- and chloroarenes, PCBs etc.), and also phenolic compounds (bromo-, chlorophenols and naphthols), first forming compounds of dehalogenation then of opening of the aromatic ring (dicarboxylic acids) up to the highest degree of oxidation (carbon dioxide and water), of organic compounds such as PCA/PAHs (aromatic polynuclears), of persistent environmental pollutants (POPs) such as PCBs (polychlorinated biphenyls), PCTs (polychlorinated terphenyls), PCBTs (polychlorinated benzyl toluenes), PCDFs (polychlorinated dibenzofurans), PCDDs (polychlorinated dibenzodioxins), PBBs (polybromated biphenyls), in addition to specific halogenated derivatives such as DDT (dichlorodiphenyltrichloroethane), dichlorodiphenyldichloroethylene (DDE), lindane and various weed killers.

The preferred oxidising agents according to the invention are sodium perborate monohydrate, sodium percarbonate (2Na₂CO₃.3H₂O₂), potassium percarbonate, barium percarbonate, potassium monoperoxysulphate, magnesium peroxide, zinc peroxide, calcium peroxide and potassium superoxide, whereas transition metal salts, in particular ferrous salts, can be appropriately used as catalysts.

The process of the invention can be implemented indiscriminately with only one of the energy sources indicated (US, MW or UV). Nevertheless the use of two of the sources is preferable and the use of all three sources mentioned even more preferable. Typically the microwaves are at 2450 M Hz and a range of power between 5 and 7000 Watts, the ultrasounds have a frequency between 18 and 500 kHz and power density from 10 to 250 Watts/cm² and the ultraviolet rays have a frequency of 200-350 nm.

The working temperature may vary from ambient temperature to 100° C.

The oxidising reagents can be used dry or mixed with the contaminated solid matrices in suspension or solution in aqueous or organic liquid matrices in addition to hydrocarbons, mineral oils, polymer oils and oils obtained from pyrolytic processes.

Examples of matrices that can be advantageously treated with the process of the invention include soil or building material, active carbon, zeolites, activated and non-activated alumina, silica and silicates, fuller's earth, pumice, isolating liquids, mineral oils, technical fluids, oily or hydrocarbon suspensions, oil from pyrolysis of polymeric materials or biomass, gaseous or liquid effluent in processes of cleaning gaseous emissions (dry or wet scrubber) of plants for pyrolysis, combustion and/or incineration and treatment of organic materials and waste in general, paper and textile waste, hospital biological material (solid and liquid) both for the purpose of its complete oxidative demolition and as pre-treatment for decontaminating the pathogenic bacteria content.

The process can be performed continuously or discontinuously, both in situ or by removal of the material to be treated.

The application in situ, in the case of contaminated surfaces or soil, involves an energetic mechanical action of remixing, performed for example with high-speed disc ploughs. Advantageously an appropriately dimensioned mobile integrated system is used, with which the soil that has been removed and broken up is simultaneously impregnated with a solution of oxidising agents and catalysts nebulised with US and radiated with MW and/or UV. Fenton's reagent can if necessary be associated with other reagents. The treatment, in the case of agricultural soil and/or an industrial area, can be at the surface or at depth (to a maximum of 40-50 cm).

The process may also comprise the recovery from the matrices of organometallic compounds or of noble and/or heavy metals, possibly after the addition of chelants, acids and/or coadjuvants: in this case the US aid in disaggregation by increasing the surface of contact for the mineralization and extraction thanks to the action of the MW.

The process of the invention allows a reduction in the treatment times, considerably improving the efficiency of the degradation process, at low matrix temperatures (typically <80-100° C.) without creating risk conditions for the work force, public health and the environment and meeting safety and application requirements.

The invention is illustrated in greater detail in the following examples and drawings.

The drawing illustrates a diagram of a multifunction reactor wherein reference numeral 1) denotes the section of radiation with MW and UV, (2) denotes the UV and US section at 20 kHz (3) and 300 kHz (4), (5) denotes a recirculation pump and (6) denotes a stirrer.

### EXAMPLE

### Treatment of 4.5 Litres of Mixed Matrix Sample (Hydrocarbons/Water) Contaminated by 3,4-Dichlorophenol (1.1 g/litre)

Approximately 50 g of sodium percarbonate (Na₂CO₃.1.5H₂O₂) were added to the mixture to be treated, or approximately 10 equivalent molars in relation to the total quantity of 3,4-dichlorophenol and 1.5 g of ferrous sulphate heptahydrate. The adding is carried out directly in the steel tank with mechanical stirrer actuated. After approximately one minute the pump, which distributes the mixture throughout the circuit, is started up and simultaneously the sonotrode is actuated at 20 kHz (80 W/cm²). Having actuated the dehumidified air cooling system, the source of MW (800 W), the sonotrode at 300 kHz and UV lamp (254 nm) are actuated. The reaction is monitored by TLC and the final sample by GC. After 8 minutes of treatment the 3,4-dichlorophenol is present in traces.

Table of comparison of the data relating to the process in question and degradation with Fenton's reagent and conventional heating on POPs in a mixed matrix (100 ml of hydrocarbons/water suspension).

| | | Innovative Process | | Fenton (100° C.) | |
|---|---|---|---|---|---|
| | | time | degrada- | | degrada- |
| | | (min- | tion | time | tion |
| Ref. | Compound | utes) | % | (hours) | % |
| 1 | 3,4-dichlorophenol | 3 | 100 | 8 | 57 |
| 2 | 2,4-dichlorotoluene | 4 | 100 | 8 | 25 |
| 3 | 1,2,4-trichlorobenzene | 4 | 100 | 8 | 13 |
| 4 | 2,4-dibromophenol | 3 | 100 | 8 | 68 |
| 5 | 4-chloro-m-cresol | 3 | 100 | 8 | 16 |
| 6 | 4-chloronaphthol | 2 | 100 | 8 | 45 |
| 7 | PCBs | 5 | 95-100 | 12 | 24 |

(10 Equiv. of Oxidisiing Agent)

The percentage of degradation was calculated by gas chromatography (FID or ECD detector).

1. Process for the degradation, detoxification and decontamination treatment of contaminated solid or liquid matrices and/or the relative functional revalorisation and for their sterilisation, which comprises placing said matrix in contact with at least one saline catalyst and at least one oxidising agent chosen from among percarbonates, perborates or metal peroxides in the presence of at last one source of energy chosen from among microwaves (MW), ultrasounds (US) and ultraviolet rays (UV).

2. Process according to claim 1 wherein the matrices are contaminated by persistent organic compounds.

3. Process according to claim 2 wherein the persistent organic compounds comprise PCA/PAHs, PCB, PCT, PCBT, PCDD, PCDF, PBB, DDT, DDE.

4. Process according to claim 1 wherein the oxidising agents are chosen from among sodium perborate monohydrate, sodium percarbonate (2Na₂CO₃.3H₂O₂), potassium percarbonate, barium percarbonate, potassium monoperoxysulphate, magnesium peroxide, zinc peroxide, calcium peroxide and potassium superoxide.

5. Process according to claim 1 wherein the catalysts are transition metal salts.

6. Process according to claim 5 wherein the catalysts are ferrous salts.

7. Process according to claim 1 wherein two sources of energy are applied simultaneously, chosen from among ultrasounds, microwaves and ultraviolet rays.

8. Process according to claim 1 wherein ultrasounds, microwaves and ultraviolet rays are applied simultaneously.

9. Process according to claim 1, characterised in that the operation is performed at a temperature between ambient temperature and 100° C.

10. Process according to claim 1 wherein the microwaves (2450 MHz) operate in a range of power between 5 and 7000 Watts, the ultrasounds have a frequency between 18 and 500 kHz and density of power from 10 to 250 Watt/cm² and the ultraviolet rays have a frequency of 200-350 nm.

11. Process according to claim 1 wherein the oxidising reagents are used dry or mixed with contaminated solid matrices, in suspension or in solution in organic or aqueous liquid matrices, hydrocarbons, or mineral, polymer or pyrolysis oils.

12. Process according to claim 1 wherein the matrices to be treated comprise soil or building material, active carbon, zeolites, activated or non-activated alumina, soil, silica and silicates, fuller's earth, pumice, isolating liquids, mineral oils, technical fluids, oily or hydrocarbon suspensions, oil from pyrolysis of polymer materials or biomass, gaseous or liquid effluent in the processes of cleaning of gaseous emissions (dry or wet scrubber) of plants for pyrolysis, combustion and/or incineration and treatment of organic materials and waste in general, paper and textile waste, hospital biological material.

13. Process according to claim 1, characterised in that it is performed continuously or discontinuously, in situ or by removal of the material to be treated.

14. Process according to claim 1, also comprising the recovery from the matrices of organometallic compounds or noble and/or heavy metals, possibly after the addition of chelants, acids and/or coadjuvants.
